(19) United States
(12) Patent Application Publication
(10) Pub. No.: US 2025/0122157 A1
(43) Pub. Date: Apr. 17, 2025

(54) TRIAZOLE-CONTAINING DEOXYBENZOIN COMPOUNDS AND POLYMERS PREPARED THEREFROM

(71) Applicant: UNIVERSITY OF MASSACHUSETTS, Westborough, MA (US)

(72) Inventors: Todd Emrick, South Deerfield, MA (US); Krishnamurthy Munusamy, Amherst, MA (US)

(21) Appl. No.: 18/910,843

(22) Filed: Oct. 9, 2024

Related U.S. Application Data
(60) Provisional application No. 63/544,294, filed on Oct. 16, 2023.

(51) Int. Cl. C07D 249/04; C08K 5/3472

(52) U.S. Cl. CPC C07D 249/04; C08K 5/3472

(57) ABSTRACT

Triazole-containing deoxybenzoin compounds are described herein. The compounds can exhibit a desirable range of thermal properties pertaining to low heat release and low flammability. In a further advantageous feature, the compounds can be used to provide triazole-containing deoxybenzoin polymers.

### CROSS-REFERENCE TO RELATED APPLICATION

This application claims priority to and the benefit of U.S. Provisional Patent Application No. 63/544,294, filed on Oct. 16, 2023, the contents of which is hereby incorporated by reference in its entirety.

### FEDERAL RESEARCH STATEMENT

This invention was made with government support under grant number 17-G-012 awarded by the Federal Aviation Administration. The government has certain rights in the invention.

### BACKGROUND

Polymers are a mainstay of modern society, for example, widely used in fabricating textiles, upholstery, construction materials, various air, land or sea vehicles, and microelectronic devices and appliances. The inherent flammability of many polymers poses a significant threat, especially in enclosed or isolated spaces. Therefore, as synthetic polymers are used extensively in society as plastics, rubbers, and textiles, polymer flammability has been recognized as a safety hazard and remains an important challenge in polymer research.

Flame retardancy of polymers is often achieved by blending polymers with flame retardant additives, such as halocarbons, including polybrominated diphenyl ether (PBDE), phosphorus, organophosphates, and metal oxides. While small molecule flame retardant additives provide a convenient means for reducing flammability of polymers, these additives can compromise safety from environmental and health perspectives. Conventional flame retardants are small molecule additives that often leach out of the polymer during their use leading to a variety of serious health and environmental problems associated with toxicity and bioaccumulation. These concerns have led to an emphasis on non-halogenated flame retardants in recent years. However, non-halogenated flame retardant additives, such as alumina trihydrate, compromise the physical and mechanical properties of polymers when loaded at high levels.

An ideal low-flammable polymer would be halogen-free and possess high thermal stability, low heat of combustion, and a low combustion heat release rate (HRR), with minimal release of toxic fumes. Intrinsically fire-resistant polymers that undergo significant carbonization upon heating are highly desirable, as carbonaceous char formation effectively averts combustion by producing an insulating layer on the polymer surface. Such char formation can also be realized from composite materials in which an additive ultimately provides the desired char.

Deoxybenzoin moieties have demonstrated utility as flame retardant materials, for example when incorporated in polyarylates, e.g., polyarylates based on 4,4′-bishydroxydeoxybenzoin (BHDB), as a bisphenolic monomer. Such polymers exhibited low combustion heat release rate and total heat of combustion, which is believed to arise from the thermally-induced conversion of BHDB to diphenylacetylene moieties that char by aromatization. See, K. A. Ellzey, T. Ranganathan, J. Zilberman, E. B. Coughlin, R. J. Farris, T. Emrick, Macromolecules 2006, 39, 3553. Pyrolysis combustion flow calorimetry (PCFC), an oxygen consumption technique for measuring heat release capacity (HRC), revealed exceptionally low HRC values for the BHDB-polyarylates (<100 J/g-K). See, R. N. Walters, M. Smith, and M. R. Nyden, International SAMPE Symposium and Exhibition 2005, 50, 1118.

There remains a continuing need in the art for new deoxybenzoin-containing materials that can provide materials for applications requiring low heat release and low flammability without the need for halogenated or other flame-retardant additives.

### SUMMARY

A deoxybenzoin compound has the structure (I), (II), (III), (IV), or (V)

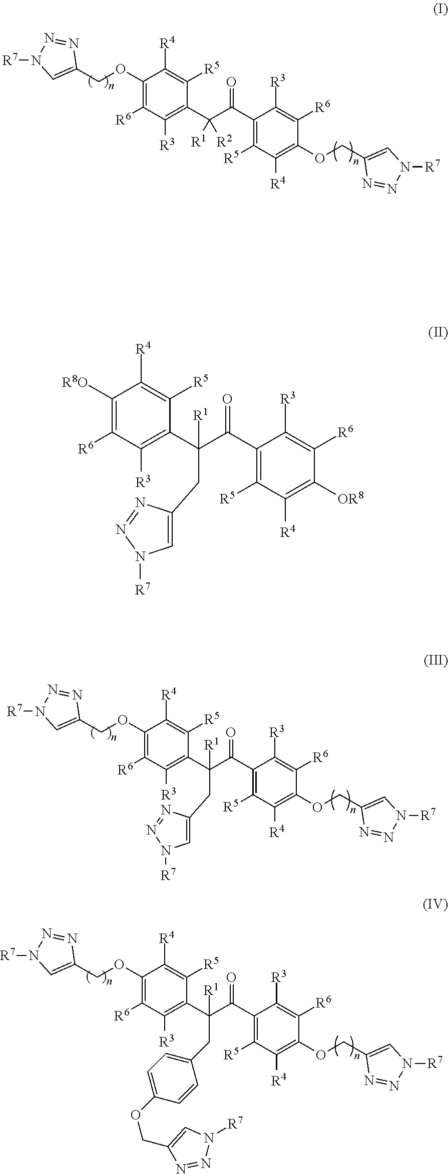

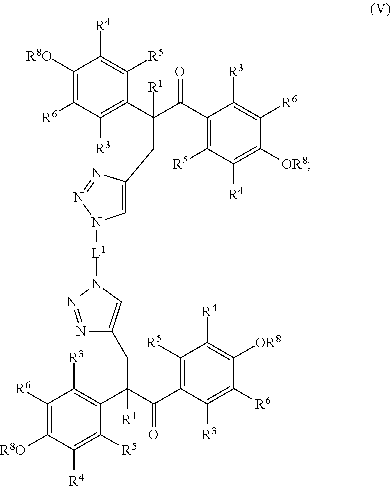

wherein in the foregoing formulas: R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; R⁷ is independently at each occurrence a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R⁸ is independently at each occurrence hydrogen or a C₁₋₆ alkyl group; L¹ is a linking group; and n is independently at each occurrence an integer from 1 to 12.

A composition comprises the deoxybenzoin compound, preferably wherein the composition comprises a thermoplastic polymer; more preferably wherein the composition comprises: 0.01 to 50 weight percent of the deoxybenzoin compound; and 50 to 99.99 weight percent of the thermoplastic polymer.

A polymer comprises repeating units of the formula

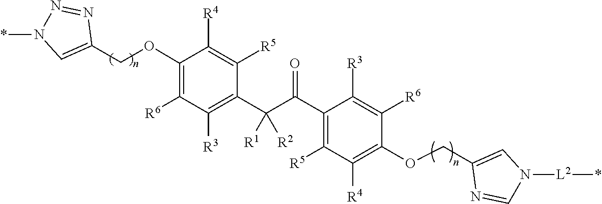

wherein R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; L² is independently at each occurrence a linking group; and n is independently at each occurrence an integer from 1 to 12.

An article comprises the polymer or the composition comprising the deoxybenzoin compound.

The above described and other features are exemplified by the following figures and detailed description.

### BRIEF DESCRIPTION OF THE DRAWINGS

The following figures represent exemplary embodiments.

FIG. 1 is a schematic illustration of synthetic pathways to yield bis-alkynyl deoxybenzoin (BADB) and tetrahydroxy deoxybenzoin triazole (THDBT) starting from desoxyanisoin.

FIG. 2 illustrates the synthesis of deoxybenzoin triazole (DBT) polymers denoted P1-P5.

FIG. 3 shows thermogravimetric analysis (TGA) (a) and microscale combustion calorimetry (MCC) thermograms (b) of tetramethoxy deoxybenzoins triazole (TMDBT), THDBT and DBT.

FIG. 4 shows MCC (a) and TGA (b) thermograms of polymers P1-P5.

FIG. 5 shows MCC thermograms of THDBT as blends with (a) polyethylene terephthalate (PET); (b) polybutylene terephthalate (PBT); (c) polyvinyl pyridine (PVP); and (d) polystyrene (PS).

FIG. 6 shows scanning electron microscope (SEM) images of char residues obtained following vertical burn experiments: (a) PET; (b) PET with 30 wt % THDBT; (c) and (d) PET with 50 wt % THDBT.

### DETAILED DESCRIPTION

The present inventors have prepared new derivatives of deoxybenzoin containing triazole moieties. The deoxybenzoin compounds including triazole moieties were prepared from the corresponding alkyne-containing deoxybenzoin compounds using azide-alkyne cycloaddition chemistry (also referred to as “click chemistry”) to yield oligomeric and polymeric structures that exhibit useful thermal properties. In particular, the triazole-containing deoxybenzoin compounds described herein advantageously offer a desirable combination of low heat release capacity (e.g., less than 100 J/g-K), high thermal stability, and high char yield. In another advantageous feature, utilizing the alkyne-substituted deoxybenzoins in azide-alkyne cycloaddition reactions gave access to deoxybenzoin-based polymers with improved solubility and processability relative to prior versions of linear, deoxybenzoin-based polymers. Moreover, the triazole units resulting from the cycloaddition reactions further contribute to their low flammability. Thus, the deoxybenzoin derivatives described herein are expected to contribute to improved flame-retardant performance of a material including these deoxybenzoin compounds. A significant improvement is therefore provided by the present disclosure.

Accordingly, an aspect of the present disclosure is a triazole-containing deoxybenzoin compound. The deoxybenzoin compound of the present disclosure has the structure (I), (II), (III), (IV), or (V)

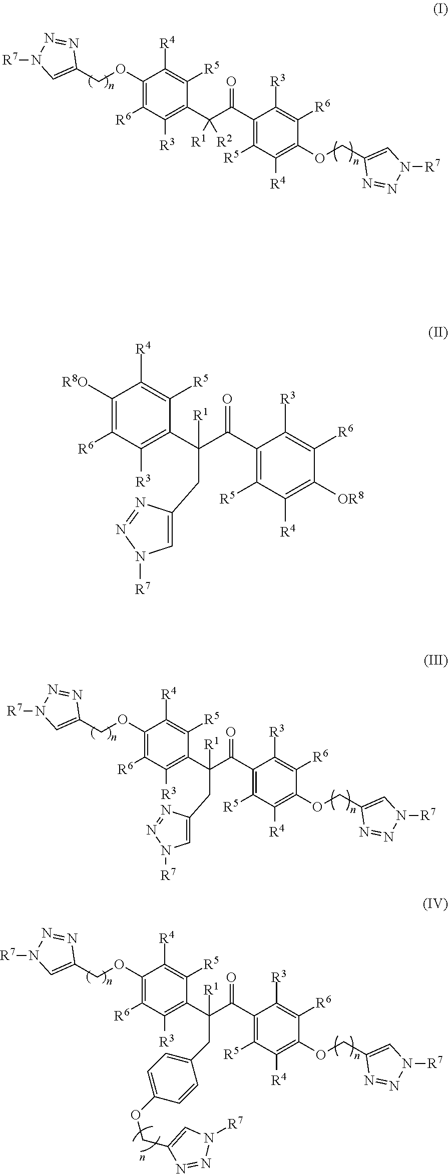

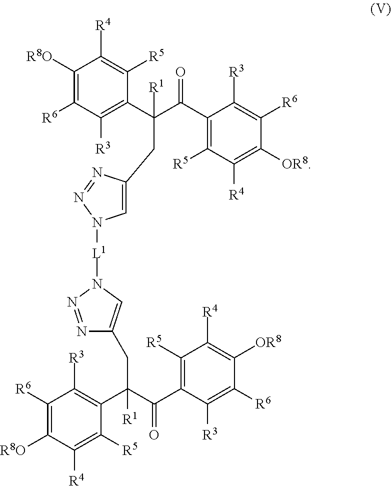

In the foregoing formulas, R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; R⁷ is independently at each occurrence a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R⁸ is independently at each occurrence hydrogen or a C₁₋₆ alkyl group; L¹ is a linking group; and n is independently at each occurrence an integer from 1 to 12.

In some aspects, each occurrence of R¹ and R² is hydrogen. In some aspects, each occurrence of n is 1. In some aspects, each occurrence of R³, R⁴, R⁵, and R⁶ is hydrogen. In some aspects, each occurrence of R⁷ is C₇₋₁₃ alkylaryl group, preferably a benzyl group. In some aspects, each occurrence of R⁸ is hydrogen or a methyl group.

In an aspect, the deoxybenzoin compound can have the structure (I), preferably wherein each occurrence of R¹ and R² is hydrogen; each occurrence of R³, R⁴, R⁵, and R⁶ is hydrogen; each occurrence of n is 1; and R⁷ is C₇₋₁₃ alkylaryl group, preferably a benzyl group. For example, the deoxybenzoin compound can have the structure (IA)

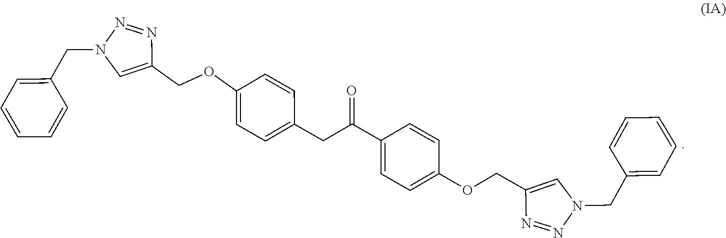 [SMILES: O=C(Cc1ccc(OCc2cn(Cc3ccccc3)nn2)cc1)c1ccc(OCc2cn(Cc3ccccc3)nn2)cc1]

In an aspect, the deoxybenzoin compound can have the structure (II), preferably wherein R¹ is hydrogen; each occurrence of R³, R⁴, R⁵, and R⁶ is hydrogen; R⁷ is C₇₋₁₃ alkylaryl group, preferably a benzyl group; and R⁸ is hydrogen or methyl. For example, the deoxybenzoin compound can have the structure (IIA) or (IIB)

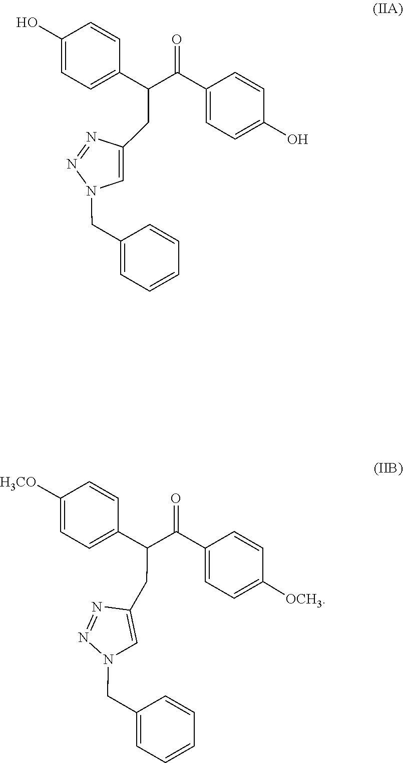 [SMILES: O=C(c1ccc(O)cc1)C(Cc1cn(Cc2ccccc2)nn1)c1ccc(O)cc1 ; COc1ccc(C(=O)C(Cc2cn(Cc3ccccc3)nn2)c2ccc(OC)cc2)cc1]

In an aspect, the deoxybenzoin compound can have the structure (III), preferably wherein R¹ is hydrogen; each occurrence of R³, R⁴, R⁵, and R⁶ is hydrogen; each occurrence of R⁷ is C₇₋₁₃ alkylaryl group, preferably a benzyl group; and n is 1. For example, the deoxybenzoin compound can have the structure (IIIA)

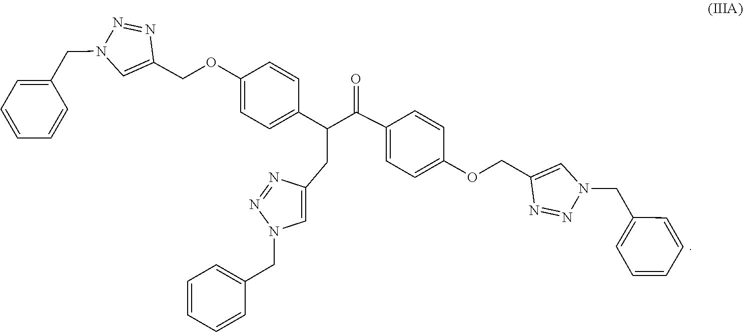

In an aspect, the deoxybenzoin compound can have the structure (IV), preferably wherein R¹ is hydrogen; each occurrence of R³, R⁴, R⁵, and R⁶ is hydrogen; each occurrence of R⁷ is C₇₋₁₃ alkylaryl group, preferably a benzyl group; and n is 1. For example, the deoxybenzoin compound can have the structure (IVA)

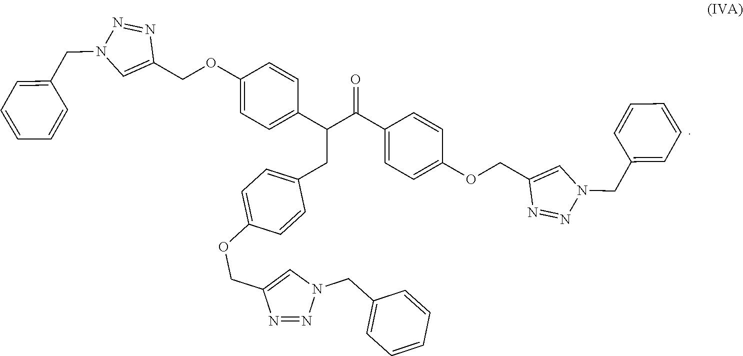 [SMILES: O=C(c1ccc(OCc2cn(Cc3ccccc3)nn2)cc1)C(Cc1ccc(OCc2cn(Cc3ccccc3)nn2)cc1)c1ccc(OCc2cn(Cc3ccccc3)nn2)cc1]

In an aspect, the deoxybenzoin compound can have the structure (V), preferably wherein each occurrence of R¹ is hydrogen; each occurrence of R³, R⁴, R⁵, and R⁶ is hydrogen; R⁸ is hydrogen or methyl; and L¹ is a C₁₋₆ alkylene group, a C₆₋₂₀ arylene group, a C₇₋₁₃ arylalkylene group, or a C₇₋₁₃ alkylarylene group. In an aspect, L¹ can be a —CH₂-Ph-CH₂— group, wherein Ph represents a phenylene group. For example, the deoxybenzoin compound can have the structure (VA) or (VB)

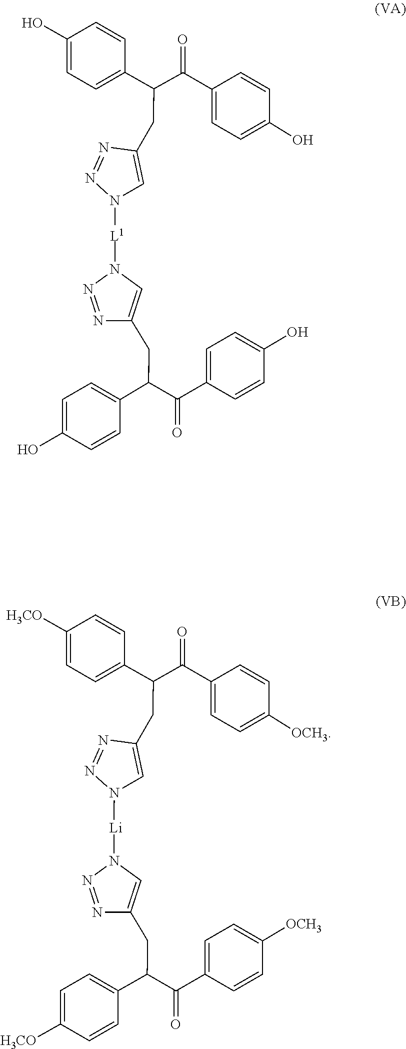

The triazole-containing deoxybenzoin compounds can be prepared from the corresponding alkyne-functionalized deoxybenzoin compounds. Exemplary alkyne-functionalized deoxybenzoin compounds that can provide the triazole-containing deoxybenzoin compounds of the present disclosure can include compounds according to formulas (VI)-(VIII)

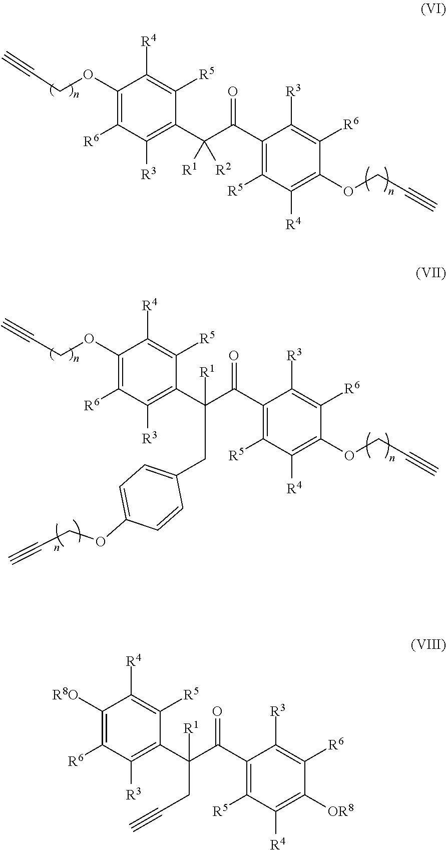

wherein R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; R⁸ is independently at each occurrence hydrogen or a C₁₋₆ alkyl group; and n is independently at each occurrence an integer from 1 to 12. In some aspects, each occurrence of R¹ and R² is hydrogen. In some aspects, each occurrence of n is 1. In some aspects, each occurrence of R³, R⁴, R⁵, and R⁶ is hydrogen. In some aspects, each occurrence of R⁸ is hydrogen or a methyl group.

Accordingly, another aspect of the present disclosure is directed to the alkyne-containing deoxybenzoin compounds according to formulas (VI)-(VIII), and in particular the compounds according to formulas (VII) and (VIII).

The method of making the triazole-containing deoxybenzoin compounds can comprise contacting an alkyne-functionalized deoxybenzoin compound with an azide-containing compound under conditions effective to provide the triazole-containing deoxybenzoin compound.

The azide-containing compound is an organic compound comprising at least one azide group (e.g., —N₃) bound thereto. In some aspects, an azide-containing compound comprising two (or more) azide groups may be used. Benzyl azide and 1,4-azidomethyl benzene are specifically mentioned.

Suitable reaction conditions including solvents, catalysts, and reaction time and temperature can be selected by the skilled person guided by the present disclosure. In general any organic solvent can be selected provided that the solvent is capable of solubilizing the components of the reaction and will not interfere in the reaction (i.e., with the reaction catalyst). An exemplary synthesis is further described in the working examples below.

The triazole-containing deoxybenzoin compounds can exhibit one or more advantageous properties, for example one or more advantageous thermal properties. For example, the deoxybenzoin compound can exhibit low heat release and low flammability properties, in part as a consequence of the high char yield obtained during their combustion.

In an aspect, the triazole-containing deoxybenzoin compounds according to the present disclosure can exhibit a heat release capacity (HRC) of less than or equal to 325 joules per gram-Kelvin, or less than or equal to 250 joules per gram-Kelvin, or less than or equal to 150 joules per gram-Kelvin, or less than or equal to 100 joules per gram-Kelvin, for example 15 to 100 joules per gram-Kelvin, determined using a pyrolysis combustion flow calorimeter. In an aspect, the triazole-containing deoxybenzoin compounds according to the present disclosure can exhibit a total heat release (THR) of less than 20 kilojoules per gram, or less than 15 kilojoules per gram, or less than 10 kilojoules per gram, for example 5 to 20 kilojoules per gram, or 5 to 15 kilojoules per gram, determined using a pyrolysis combustion flow calorimeter. In an aspect, the triazole-containing deoxybenzoin compounds according to the present disclosure can exhibit a char yield of at least 20 percent, for example 20 to 60 percent, after 60 minutes at 800° C., as determined by thermogravimetric analysis. In an aspect, the triazole-containing deoxybenzoin compounds according to the present disclosure can exhibit a fire growth capacity (FGC) of less than 150 joules per gram-Kelvin. In an aspect, the triazole-containing deoxybenzoin compounds can exhibit one or more of the foregoing properties.

The desirable thermal properties exhibited by the triazole-containing deoxybenzoin compounds can make them well-suited for use as a flame retardant additive in a composition. Thus a composition, for example a polymer composition, comprising the triazole-containing deoxybenzoin compound represents another aspect of the present disclosure.

In an aspect, the composition can comprise a thermoplastic polymer and the deoxybenzoin compound. As used herein, the term “thermoplastic” refers to a material that is plastic or deformable, melts to a liquid when heated, and freezes to a brittle, glassy state when cooled sufficiently. Thermoplastics are typically high molecular weight polymers. Examples of thermoplastic polymers that can be used include polyacetals (e.g., polyoxyethylene and polyoxymethylene), poly(C₁₋₆ alkyl)acrylates, polyacrylamides, polyamides, (e.g., aliphatic polyamides, polyphthalamides, and polyaramides), polyamideimides, polyanhydrides, polyarylates, polyarylene ethers (e.g., polyphenylene ethers), polyarylene sulfides (e.g., polyphenylene sulfides), polyarylsulfones, polybenzothiazoles, polybenzoxazoles, polycarbonates (including polycarbonate copolymers such as polycarbonate-siloxanes, polycarbonate-esters, and polycarbonate-ester-siloxanes), polyesters (e.g., polyethylene terephthalates, polybutylene terephthalates, polyarylates, and polyester copolymers such as polyester-ethers), polyetheretherketones, polyetherimides (including copolymers such as polyetherimide-siloxane copolymers), polyetherketoneketones, polyetherketones, polyethersulfones, polyimides (including copolymers such as polyimide-siloxane copolymers), poly(C₁₋₆ alkyl)methacrylates, polymethacrylamides, polynorbornenes (including copolymers containing norbornenyl units) polyolefins (e.g., polyethylenes, polypropylenes, polytetrafluoroethylenes, and their copolymers, for example ethylene-alpha-olefin copolymers), polyoxadiazoles, polyoxymethylene, polyphthalides, polysilazanes, polysiloxanes, polystyrenes (including copolymers such as acrylonitrile-butadiene-styrene (ABS) and methyl methacrylate-butadiene-styrene (MBS)), polysulfides, polysulfonamides, polysulfonates, polysulfones, polythioesters, polytriazines, polyureas, polyurethanes, polyvinyl alcohols, polyvinyl esters, polyvinyl ethers, polyvinyl halides, polyvinyl ketones, polyvinyl thioethers, polyvinylidene fluorides, or the like, or a combination comprising at least one of the foregoing thermoplastic polymers. Polyacrylates, polyurethanes, and polyimides may be especially useful in a wide variety of articles. Combinations of any of the foregoing thermoplastic polymers may be used.

The composition can comprise the deoxybenzoin compound in an amount effective to provide a desired thermal effect for the composition. For example, in an aspect, the composition can comprise 0.01 to 50 weight percent of the deoxybenzoin compound, based on the total weight of the composition. Within this range, a composition can comprise 0.01 to 40 weight percent, or 0.01 to 30 weight, or 0.1 to 30 weight percent, or 0.1 to 25 weight percent, or 1 to 30 weight percent, or 1 to 25 weight percent, each based on the total weight of the composition. Conversely, the composition can comprise 50 to 99.99 weight percent of a thermoplastic polymer, based on the total weight of the composition. Within this range, the thermoplastic polymer can be present in an amount of 60 to 99.99 weight percent, or 70 to 99.99 weight percent, or 75 to 99.9 weight percent, or 70 to 99 weight percent, or 75 to 99 weight percent, each based on the total weight of the composition.

Other additives that are generally known in the art may be present in the composition provided that they do not significantly adversely affect one or more desired properties of the composition (e.g., thermal properties). Such additives can include, but are not limited to, stabilizers, mold release agents, lubricants, processing aids, drip retardants, nucleating agents, UV blockers, dyes, pigments, antioxidants, anti-static agents, blowing agents, mineral oil, metal deactivators, antiblocking agents, and combinations thereof. When present, such additives are typically used in a total amount of less than or equal to 5 weight percent, specifically less than or equal to 2 weight percent, more specifically 0.01 to 2 weight percent, based on the total weight of the composition. It will be understood the individual amounts of each component present in the composition sum to a total of 100 weight percent.

Another aspect of the present disclosure is a triazole-deoxybenzoin-containing polymer comprising repeating units derived from reaction of an alkyne-containing deoxybenzoin and an azide-containing compound. As will be recognized by the skilled person, in order to provide the polymer, the alkyne-containing deoxybenzoin compound must contain at least two alkyne groups. Similarly, the azide-containing compound must contain at least two azide groups. In an aspect, the alkyne-containing deoxybenzoin compound contains two alkyne groups and the azide-containing compound contains two azide groups. It will be understood that when more than two functional groups are present on either of the alkyne-containing deoxybenzoin or the azide compound, a branched polymer material will be the result. In some cases, a crosslinked polymer may result. Exemplary alkyne-functionalized deoxybenzoin compounds and azide compounds can be as discussed above.

In an aspect, the polymer comprises repeating units of the formula

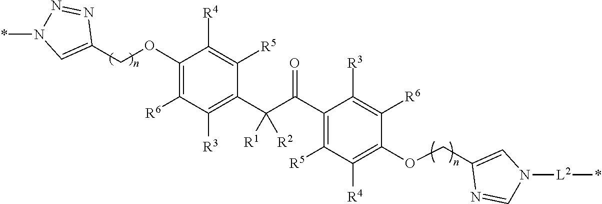

wherein R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; L² is independently at each occurrence a linking group; and n is independently at each occurrence an integer from 1 to 12. The asterisks (*) indicate points of attachment of the repeating unit to the rest of the polymer backbone.

In some aspects, each occurrence of R¹ and R² is hydrogen. In some aspects, each occurrence of n is 1. In some aspects, each occurrence of R³, R⁴, R⁵, and R⁶ is hydrogen. In some aspects, each occurrence of R⁷ is C₇₋₁₃ alkylaryl group, preferably a benzyl group. In some aspects, each occurrence of R⁸ is hydrogen or a methyl group. In some aspects, L² is a C₁₋₆ alkylene group, a C₆₋₂₀ arylene group, a C₇₋₁₃ arylalkylene group, or a C₇₋₁₃ alkylarylene group. In an aspect, L² can be a —CH₂-Ph-CH₂— group, wherein Ph represents a phenylene group.

The polymer can be prepared by contacting an alkyne-containing deoxybenzoin compound with an azide compound, as described above, under conditions effective to provide the polymer. Suitable reaction conditions including solvents, catalysts, and reaction time and temperature can be selected by the skilled person guided by the present disclosure. In general any organic solvent can be selected provided that the solvent is capable of solubilizing the components of the reaction and will not interfere in the reaction (i.e., with the reaction catalyst). An exemplary synthesis is further described in the working examples below.

In an aspect, the polymer can have a number average molecular weight of 1,000 to 500,000 grams per mole (g/mol), as determined by gel permeation chromatography. In an aspect, the polymer can have a number average molecular weight of 10,000 to 500,000 g/mol.

The polymer according to the present disclosure can exhibit one or more desirable properties, for example a particular set of thermal properties. For example, the polymer can exhibit low heat release and low flammability properties, in part as a consequence of the high char yield obtained during their combustion.

In an aspect, the polymer according to the present disclosure can exhibit a heat release capacity (HRC) of less than or equal to 150 joules per gram-Kelvin, or less than or equal to 100 joules per gram-Kelvin, or less than or equal to 85 joules per gram-Kelvin, or less than or equal to 80 joules per gram-Kelvin, for example 50 to 100 joules per gram-Kelvin, determined using a pyrolysis combustion flow calorimeter. In an aspect, the polymer according to the present disclosure can exhibit a total heat release (THR) of less than 20 kilojoules per gram, or less than 15 kilojoules per gram, or less than 10 kilojoules per gram, for example 5 to 10 kilojoules per gram, determined using a pyrolysis combustion flow calorimeter. In an aspect, the polymer according to the present disclosure can exhibit a char yield of at least 20 percent, for example 20 to 60 percent, after 60 minutes at 800° C., as determined by thermogravimetric analysis. In an aspect, the polymer according to the present disclosure can exhibit a fire growth capacity (FGC) of less than 65 joules per gram-Kelvin, or less than 60 joules per gram-Kelvin, or less than 55 joule per gram-Kelvin, or less than 50 joules per gram-Kelvin, for example 40 to 55 joules per gram-Kelvin or 40 to 50 joules per gram-Kelvin. In an aspect, the polymer can exhibit one or more of the foregoing properties.

The compounds and polymers and the synthetic methodologies disclosed herein can have broad impact on such diverse fields of fabricating textiles, upholstery, construction materials, various air, land or sea vehicles, and microelectronic devices and appliances. Thus the deoxybenzoin compounds and polymers prepared therefrom as described above can be particularly useful for the manufacture of various articles, specifically where improved thermal properties or flame retardance is desired. In some aspects, the article can be a film, a coating (e.g., a UV-curable coating), a fiber, a textile, a furniture component, construction materials (e.g., insulation), a vehicle component (e.g., an automobile component, a railway vehicle component, a marine vehicle component, an airplane component, and the like), an electronic component, an adhesive, or a foam.

This disclosure is further illustrated by the following examples, which are non-limiting.

### EXAMPLES

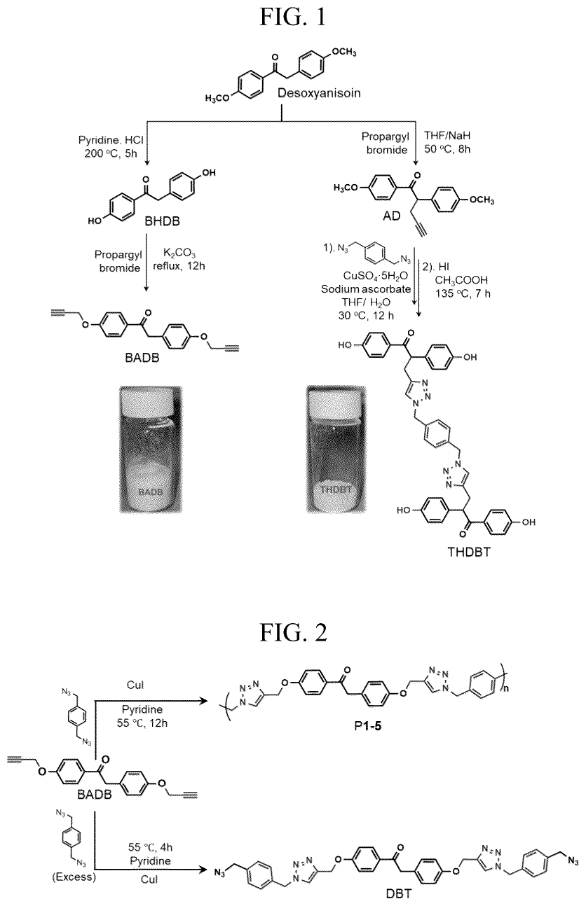

As illustrated in FIG. 1, starting from the commercially available desoxyanisoin, alkynyl substitution was performed via the two synthetic pathways shown, ultimately giving access to functional deoxybenzoins that may react either through their alkynyl or phenolic groups. To synthesize BADB, the commercially available desoxyanisoin was first demethylated with pyridine hydrochloride, and the resultant phenols were converted to propargyl ethers in an acetone solution of propargyl bromide in the presence of K₂CO₃. BADB was obtained as a white solid in 81% yield after crystallization from methanol and its chemical structure was confirmed by ¹H and ¹³C NMR spectroscopy. In the ¹H NMR spectrum, the characteristic alkyne C≡C—H signals appeared at 2.57 and 2.53 ppm (reflecting the asymmetry of the deoxybenzoin structure), and three baseline-separated methylene signals were found at 4.77 and 4.69 (propargyl CH₂ groups) and 4.21 ppm (CH₂ positioned alpha to ketone). The ¹³C NMR spectrum showed the expected signals for the alkynyl carbons (C≡CH) at 75.5, 76.2, 77.7 and 78.6 ppm, as well as methylene resonances at 55.8 and 44.3 ppm.

In another approach, by utilizing the acidity of the α-keto methylene group, desoxyanisoin was converted to the alkynyl derivative alkynyl desoxyanisoin (AD) by its reaction with sodium hydride in tetrahydrofuran, followed by quenching with propargyl bromide. AD was obtained as a yellow powder in 80% isolated yield after purification by precipitation into ether. In the ¹H NMR spectrum, an alkynyl proton resonance was found at 1.95 ppm and the central α-keto methyne (CH) proton appeared as a triplet at 4.77 ppm. In the ¹³C NMR spectrum, the characteristic alkyne carbon (C≡CH) resonances appeared at 70.1 and 82.8 ppm, the methoxy carbons at 55.3 and 55.0 ppm, and the methylene (—CH₂—) and methyne (—CH) carbons at 51.61 and 53.73 ppm, respectively. Subjecting AD to azide-alkyne cycloaddition with 1,4-bis(azidomethyl)benzene, catalyzed by CuSO₄·5H₂O, produced the corresponding triazole-linked tetramethoxy dimer as pale green solid in 90% yield. The disappearance of the original alkynyl ¹H NMR signal of AD, and the appearance of several new signals (benzyl methylenes at 5.42 ppm, triazole protons and aromatic protons from dibenzyl moieties at 7.05 ppm, and methoxy at 3.82 and 3.74 ppm) were indicative of successful coupling and triazole formation. In its ¹³C NMR spectrum, the cycloadduct showed the expected triazole carbon resonance at 123.1 ppm (and an absence of any alkynyl carbon resonances), and a benzylic methylene peak at 30.1 ppm, representative of the triazole product. Furthermore, MALDI-TOF analysis showed the expected molecular weight of 776.57 [M+H]⁺.

The cycloadduct was then demethylated by stirring in an acetic acid solution of hydroiodic acid (HI) at ˜135° C. for 7 h, which was isolated by precipitation into water, yielding the tetraphenolic product (THDBT) as a light-green solid in 85% yield. The ¹H NMR spectrum of THDBT confirmed the expected disappearance of the methyl ether resonances and the appearance of broad phenolic signals (the signal breadth likely a result of H-bonding with the triazole groups) at 10.33 and 9.29 ppm that integrated as expected relative to the remaining resonances. Compared to its methyl ether precursor, the characteristic triazole protons of THDBT shifted downfield to 7.66 ppm, while the ¹³C NMR spectrum confirmed demethylation through absence of the methoxy carbons, while MALDI-TOF analysis showed the expected molar mass of 720.39 [M+H]⁺.

### Cycloaddition Polymerization Using BADB

The bis-alkynyl structure of BADB makes it an excellent candidate for polymerization by cycloaddition with partner diazido comonomers to yield the corresponding triazole-containing polymer products. For example, as illustrated in FIG. 2, solution polymerization was performed using BADB and 1,4-bis(azidomethyl)benzene, employing CuI catalysis in a solution of pyridine. The polymerization mixture was stirred at 55° C. for 12 h, followed by precipitation from MeOH, rinsing of the polymer products with dichloromethane, and drying under vacuum to isolate polymers P1-P5 as off-white solids. Gel permeation chromatographic (GPC) analysis was performed in DMF (using PMMA calibration standards) to estimate polymer molecular weight, giving number-average molecular weight (Mₙ) values up to ˜10 kDa, with polydispersity index (PDI) ranging of ˜2, or higher in cases where the GPC chromatograms contained high molecular weight shoulders. Such PDI values are expected for step-growth polymerizations that often deviate from the ideal theoretical value of 2. The polymerizations were performed on gram scale and the polymer products isolated in appreciable yields (˜70%) after purification. The ¹H NMR spectrum of PI in DMSO-d⁶ exhibited triazole proton signals at 8.29 and 8.25 ppm, and the absence of alkyne signals, pointing to successful propagation by cycloaddition. In the ¹H NMR spectra of the polymeric cycloadducts, methylene proton resonances were found at 5.22, 5.07 and 5.59 ppm, while the peak at 7.32 ppm corresponds to the phenyl protons from the connecting aromatic moieties. The ¹³C NMR spectrum showed the triazole carbon resonances at 125.2 and 125.4 ppm, an absence of alkynyl carbons, and a benzylic methylene signal at 44.0 ppm. These step-growth polymerizations were performed using the range of monomer stoichiometries indicated in Table 1, to produce deoxybenzoin triazoles P2-P5, whereby increasing the proportion of BADB in the feed ratio yielded polymers with larger extents of alkyne chain ends (seen for example in the ¹H NMR spectrum); as expected, reactions employing the largest stoichiometric monomer imbalance yielded the lowest molecular weight products. When a 3:1 molar ratio of azide:alkyne monomers was employed, propagation was minimal, and the azide-terminated bis-triazole (DBT) was isolated in considerable yield. The structure of DBT was confirmed by MALDI-TOF analysis with molecular weight of 680.47 [M+H]⁺ as well as by ¹H NMR spectroscopy that showed the triazole protons resonating at 7.98-8.00 ppm as well as four distinct methylene resonances at 5.22 and 5.07 (connecting to deoxybenzoin), 5.59 ppm (N—CH₂ of triazoles), and 4.38 ppm (CH₂—N₃). DBT was found to be soluble in common organic solvents (CH₂Cl₂ and THF), noting that the reactivity of its azide chain-ends is well-situated for cycloaddition or photo-induced grafting chemistry.

TABLE 1

| | | GPC^{a} | | | |
|---|---|---|---|---|---|
| | Monomer ratios | Mₙ | M_{w} | | yield |
| Polymer | BAB:BADB | (g/mol) | (g/mol) | Ð | (%) |
| P1 | 1.1 | 8200 | 22400 | 2.7 | 78 |
| P2 | 1:1.2 | 9800 | 20800 | 2.1 | 75 |
| P3 | 1:1.5 | 6600 | 12500 | 1.9 | 74 |
| P4 | 1:2.0 | 6900 | 14800 | 2.1 | 75 |
| P5 | 1:2.5 | 3600 | 6000 | 1.7 | 70 |

^{a}Estimated by GPC analysis using DMF as the mobile phase and PMMA calibration standards.

### Thermal Properties and Heat Release Measurements

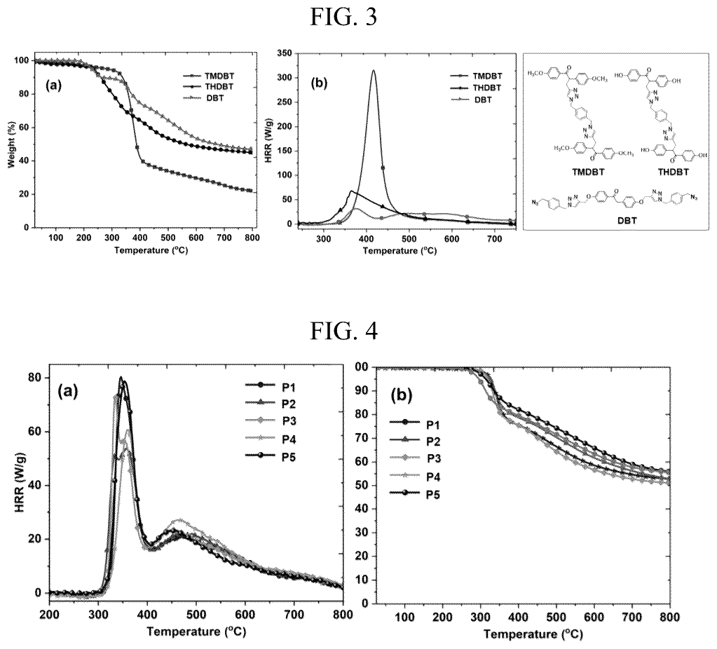

The thermal behavior and heat release properties of the molecular and macromolecular structures described above were evaluated using thermogravimetric analysis (TGA), differential scanning calorimetry (DSC) and microscale combustion calorimetry (MCC), with results of these measurements summarized in Table 2. For example, TGA analysis of TMDBT in FIG. 3(a) revealed a steep mass loss onset at ˜350° C. and a final char yield of 22%; in contrast, the tetraphenolic THDBT decomposed gradually, producing a char yield of nearly 50% at 800° C. The TGA curve of DBT displayed multiple degradation stages, attributable to elimination of N_{2(g)}, first by decomposition of the terminal azides (˜200° C.), followed by the triazole units (315-395° C.), leading to a considerable final char yield of ˜52%. DSC analysis of TMDBT showed a melting transition of ˜70° C., while the higher melting temperature of THDBT (134° C.) likely reflects the impact of phenol-triazole hydrogen bonding. The MCC experiments shown in FIG. 3(b) similarly distinguished between the methylated and demethylated structures. Data obtained from MCC analysis is useful for visualizing the heat release profiles of molecular and macromolecular structures, requiring only mg quantities of material, with results that reveal key flammability parameters of heat release capacity (HRC) and fire growth capacity (FGC). MCC measurements of THDBT gave a broad heat release profiles and an exceedingly low HRC value of ˜68 J g⁻¹K⁻¹, and an FGC of 60 J/g-K, while the sharp heat release profile and higher heat release rate observed for TMDBT corresponds to higher HRC (˜307 J g⁻¹ K⁻¹) and FGC (214 J/g-K) values. Thus, simply removing the methyl groups of TMDBT produces a large reduction in heat release and makes the tetraphenolic compound an attractive candidate for applications in both the capacity of a low flammability additive as well as that of a reactive (monomeric) precursor to low flammability polymers. DBT also exhibited exceptionally low HRC and FGC values (43 and 44 J g⁻¹ K⁻¹, respectively) owing to the presence of very little hydrocarbon fuel in this C,H,N-rich aromatic macromolecular composition.

TABLE 2

MCC data

| | FGC^{a} | HRC^{a} | THR^{a} | TGA^{b} |
|---|---|---|---|---|
| Compound | (J/g-K)) | (J/g-K) | (KJ/g) | Char yield (%) |
| TMDBT | 214 | 307 | 14.7 | 22 |
| THDBT | 61 | 69 | 8.9 | 47 |
| DBT | 43 | 44 | 7.7 | 52 |
| P1 | 47 | 77 | 6.6 | 52 |
| P2 | 45 | 58 | 6.4 | 52 |
| P3 | 47 | 80 | 6.9 | 50 |
| P4 | 48 | 66 | 6.8 | 51 |
| P5 | 47 | 74 | 7.4 | 49 |

^{a}Heat release values calculated from MCC curves;
^{b}TGA analyses were performed under nitrogen atmosphere and char residue obtained at 800° C.

Evaluation of the polymeric click cycloadducts P1-P5 (FIG. 4) similarly places all of them in the ultra-low heat release category, with MCC-derived data in Table 2 indicating FGC (˜50 J/g-K), HRC (˜50-80 J/g-K), and THR (˜7-8.5 kJ/g) values that were consistently low across these compositions; in accord with these results and their chemical structures, TGA analysis showed that consistently high char yields of about 50% for each sample (FIG. 4(b)). DSC thermograms of P1-P5 showed glass transitions (T_{g}) just above 120° C. Taken together, the thermal properties of these deoxybenzoin triazole structures are compelling for further investigation, noting that a recent Federal Aviation Administration reports describes materials with FGC values ˜50 J/g-K or less as likely to meet the flammability requirements needed for use in applications requiring ultra-low flammability; the ability to achieve such low values in the absence of halogen or phosphorus, and with no added inorganic components (e.g., fiberglass, etc.), suggests that these types of structures described will be of interest for integration into low flammability materials.

### Thermal Properties of THDBT-Polymer Blends

Noting the impressively low heat release properties of the deoxybenzoin click cycloadducts, we investigated their impact on the heat release and flammability properties of commodity polymers through blending, selecting THDBT as the test case. This was done by dissolving THDBT with the selected polymer, including poly(ethylene terephthalate) (PET), poly(butylene terephthalate) (PBT), poly(4-vinyl pyridine) (P4VP), and polystyrene (PS), followed by solvent evaporation and characterization by infrared spectroscopy and a range of thermal and flammability measurements. FT-IR spectroscopy of THDBT itself showed characteristic features for the triazole at 1442 and 1035 cm⁻¹, and for the deoxybenzoin carbonyl at 1660 cm⁻¹, while the 1712 cm⁻¹ stretching frequency of PET reflects the ester carbonyl groups, and signals at 1410 and 1243 cm⁻¹ are associated with bending vibrations of the —CH₂— and ester C(O)—O groups, respectively. In the PET-THDBT blends, the PET ester signal shifted slightly (to 1716 cm⁻¹), likely a result of H-bonding between the two components.

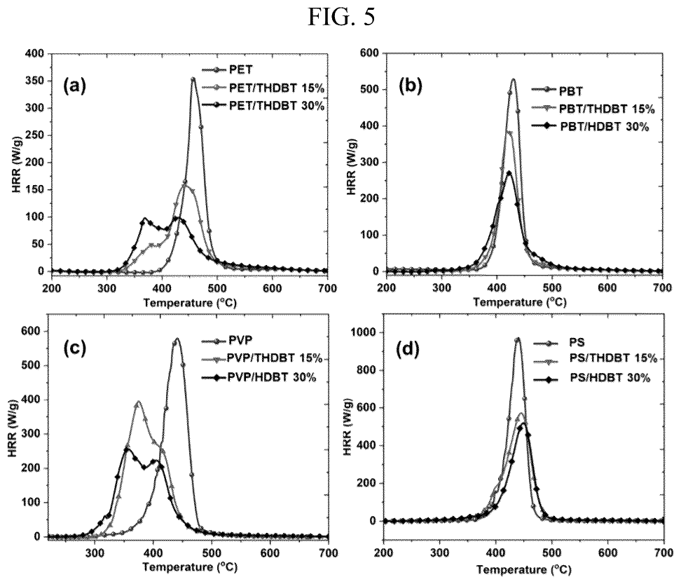

The low char yields inherent to pure PET, ˜8% at 800° C., was more than doubled in blends containing 30 wt. % THDBT. The more extensive aliphatic character of PBT makes in nearly devoid of char (char yield ˜4%), which increased significantly to 16% when blended with THDBT (again at 30 wt. %). For P4VP and PS, which essentially produce no char yield, the presence of 30 wt % THDBT increased char only minimally, by ˜8% and 4%, respectively. The results of HRC analyses these polymer-THDBT blends are shown in FIG. 5, noting that the deoxybenzoin additive had an impressively substantial impact on the HRC of PET (FIG. 5(a)), reducing HRC to ˜160 J/g-K (a 50% decrease) at 15 wt % THDBT, and to ˜99 J/g-K at 30 wt % THDBT. For PBT, the relatively high HRC (>500 J/g-K) of the pure polymer was reduced substantially with 15 and 30 wt % THDBT, to 384 J/g-K and 267 J/g-K, respectively (FIG. 5(b)). Interestingly, adding THDBT to PVP (FIG. 5(c)) led to both a decline in heat release and a significant shift of peak heat release, which is attributed to pyridine-phenol intermolecular interactions that would be expected from this polymer-additive combination. In PS, also a highly flammable and low-charring polymer, again HRC values declined (FIG. 5(d)) for the THDBT-PS blend, though notably without the peak heat release shift seen for PVP.

TGA and MCC experiments were also performed on commercially available PET reinforced with 30 wt. % glass, denoted g-PET, which as anticipated produced higher char yield (˜40%) and lower HRC values (˜210 J/g-K) relative to pure PET. Interestingly, while blending 30 wt. % THDBT into g-PET led to no significant increase in char yield, HRC values were reduced significantly, measured as ˜100 J/g-K. For comparison, PET, g-PET and PBT were blended similarly with tetrabromobisphenol A (TBBPA), which we view as a suitable halogenated comparison for its phenolic character. The performance of THDBT as an additive was remarkably similarly to TBBPA, and thus supports its potential as a non-halogenated, anti-flammable additive by virtue of its propensity to produce char rather than flammable gas, offering an alternative to halogenated structures for improving the fire safety of polymer-based materials.

### Reducing Polymer Flammability by Blending With THDBT

To further evaluate the impact of THDBT to reduce polymer flammability by blending, vertical burn tests were performed in a manner similar to the often-utilized UL-94 test that characterizes flame propagation, time to self-extinguishing, and melting/dripping properties of the test sample. Test specimens were prepared by solution casting of THDBT/polymer blend on silicone molds to give rectangular specimens of 70 mm length and 3 mm thickness. Vertical burn tests were then performed in an ambient atmosphere by applying the flame from a propane torch to the bottom of the specimen for 10 seconds. For cases where the flame extinguished on its own, the time to self-extinguish was recorded, after which the specimen was exposed to the flame for an additional 10 seconds. Most notable from the range of vertical burn experiments performed was the impact of THDBT on the flammability of PET. An ignited sample of pure PET burned without extinguishing, and dripped extensively onto underlying cotton. In contrast, PET with 50 wt % THDBT self-extinguished in ˜10 seconds after the first ignition; after a second ignition, similar self-extinguishing was observed and the charred sample maintained its shape without dripping. Using 30 wt % THDBT decreased the flammability of PET, but not to the same extent. Additionally interesting was the impact of THDBT on g-PET, in tests on 0.5 mm thick specimens. In the present experiments, the glass additive of g-PET alone was ineffective in flammability tests of this type, as the specimen burned without extinguishing, eventually breaking, and igniting the underlying cotton sample, whereas g-PET with only 25 wt % THDBT self-extinguished in ˜4 seconds after the first ignition. Similar flame-tests performed on g-PET-TBBPA blends also self-extinguished without dripping, thus reinforcing the capability of the halogen-free deoxybenzoin additive to provide impactful levels of flame-retardance that are more typically associated with halogenated structures. We note that similar vertical burn experiments on PBT, PS, and P4VP-based blends showed the THDBT additive to be less effective, as would be anticipated from HRC results, providing sufficient evidence that more complex blending formulations are needed to retard their flammability. For PBT, 30 wt % THDBT led to self-extinguishing in ˜40 seconds, notably without dripping, while the high flammability of PS and P4VP overwhelmed any potential influence of the additives.

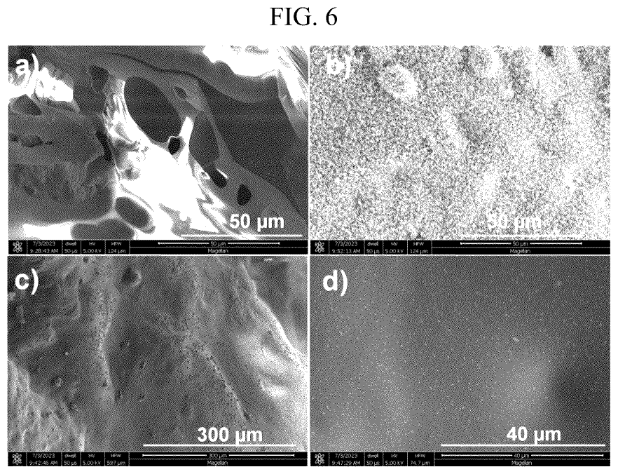

Interestingly, the surface morphologies of the char residues following vertical burn tests, analyzed by scanning electron microscopy (SEM), changed considerably between PET itself and the THDBT-PET blends, as shown in FIG. 6. The PET char was non-uniform with large pores throughout, attributable at least in part to the release of volatile gas during combustion. In contrast, for the 30% THDBT-PET blend, SEM analysis showed a much more uniform structure, lacking the large porous features, while the 50% THDBT-PET blends produced char with a relatively smooth surface. Such morphological differences may reflect the role of char structure formation in flame extinction, whereby charring of the additive aids in morphological evolution during combustion. Similarly, SEM of the charred g-PET revealed a porous morphology, whereas char from the 25% THDBT-g-PET blend had a relatively homogeneous and smooth surface.

In summary, a new set of triazole-rich deoxybenzoin compounds was synthesized from alkynyl-substituted precursors using azide-alkyne cycloaddition reactions. The resultant deoxybenzoin-containing triazoles, both as discrete molecules and as polymers, were obtained in high yields and found to exhibit desirably low heat release properties when evaluated by microscale combustion calorimetry. Unlike the low molecular weight (monomeric) deoxybenzoin itself (i.e., bis-hydroxydeoxybenzoin or BHDB), the structures according to the present disclosure proved useful as additives for promoting the fundamental science of low flammability materials and for reducing the heat release and flammability of poly(ethylene terephthalate (PET)). The deoxybenzoin-based structures described here are anticipated to be advantageous for flame-retardant solutions that avoid the potential issues associated with halogen-and phosphorus-based flame-retardants. A significant improvement is therefore provided by the present disclosure.

Experimental details follow.

Materials. The chemicals used in this work were purchased from Sigma-Aldrich, including desoxyanisoin (98%), sodium hydride (95%), propargyl bromide (80%), α,α′-Dichloro-p-xylene (98%), hydroiodic acid (57%), sodium azide, CuI, and poly(ethylene terephthalate) (PET) with 30% glass particles as a reinforcer. Pure (additive-free) PET was obtained from Eastman. Bishydroxydeoxybenzoin (BHDB) and 1,4-bis(azidomethyl)benzene were prepared according to literature procedures. Tetrahydrofuran (THF) was purchased from Fisher Scientific and was dried and distilled over sodium/benzophenone ketyl before use.

Instrumentation. Nuclear magnetic resonance (NMR) spectra were recorded using a Bruker Advance-500 spectrometer at 25° C. unless stated otherwise. Fourier Transform Infrared (FTIR) spectroscopy measurements were carried out on a Perkin Elmer Spectrum One FT-IR spectrometer equipped with an attenuated total reflectance (ATR) accessory. Matrix-assisted laser-desorption ionization time-of-flight (MALDI-TOF) mass spectrometry was carried out on Bruker MicroFlex instrument. TGA thermograms were acquired on a TA Instruments Q50 under N_{2(g)} atmosphere and operating at a 10° C./min heating rate. DSC thermograms were obtained on a TA Instruments Q20, using 10° C./min heating and cooling rates. Gel permeation chromatography (GPC) was performed on an Agilent 1260, using DMF as eluent and a 1 mL/min flow rate, with PMMA calibration standards. MCC analysis was performed according to the ASTM D7309-21, Method A, using an 80 cc/min stream of N_{2(g)} and a heating rate of 1° C./s. In the MCC instrument, the anaerobic thermal degradation products were combined with a 20 cc/min stream of oxygen gas in a furnace at 900° C. Heat release capacity (HRC) and total heat release (THR) values were calculated according to the data obtained. Vertical burning tests were performed using a procedure similar to that of the ASTM D3801 vertical burn test. Test specimens were 70 mm in length and 3 mm in thickness (in some cases, 0.5 mm thickness). These vertical burning tests employed a propane flame under atmospheric conditions, allowing the flame to contact the material for 10 seconds, then recording time to self-extinguish. The specimens were exposed to the flame for an additional 10 seconds, and the time self-extinguishing was recorded. The resultant charred samples were characterized by scanning electron microscopy on a Magellan 400 instrument equipped with a field emission gun.

Synthesis of bis-alkynyl deoxybenzoin (BADB). BHDB (6.00 g, 26.2 mmole) and acetone (240 mL) were added to a 500 mL flame-dried two-necked round-bottle equipped with a reflux condenser, nitrogen gas inlet and magnetic stir bar. Potassium carbonate (9.05 g, 65.5 mmole) and potassium iodide (4.34 mg, 0.026 mmole) were added, and the resultant solution was stirred at room temperature for 10 mins. Propargyl bromide (4.36 mL, 57.6 mmole) was then added dropwise at room temperature and the mixture stirred at reflux for 12 hours. The reaction mixture was passed through Celite, and solvent was removed by rotary evaporation. The crude product was recrystallized from MeOH and isolated as a white solid. (6.5 g, 21 mmol, 81% yield). ¹H NMR (CDCl₃, 500 MHz, 298 K): δ_{H} 8.03 (d, J=8.9 Hz, 2H, Ar H), 7.22 (d, J=8.7 Hz, 2H, Ar H), 7.04 (d, J=8.9 Hz, 2H, Ar H), 6.96 (d, J=8.7 Hz, 2H, Ar H), 4.77 (d, J=2.4 Hz, 2H, CH₂), 4.69 (d, J=2.4 Hz, 2H, CH₂), 4.21 (s, 2H,CH₂), 2.57 (t, J=2.4 Hz, 1H), 2.53 (t, J=2.4 Hz, 1H). ¹³C NMR (CDCl₃, 125 MHz, 298 K): δ_{H} 196.35, 161.32, 156.52, 130.85, 130.48, 130.36, 127.82, 115.13, 114.69, 78.65, 77.77, 76.26, 75.55, 55.86, 44.38 ppm.

Synthesis of alkynyl desoxyanisoin (AD). Desoxyanision (20.0 g, 78.0 mmole) and THF (200 mL) were added to a 500 mL flame-dried two-necked round-bottle equipped with a reflux condenser, nitrogen gas inlet, magnetic stir bar, and an addition funnel. Sodium hydride (2.34 g, 97.5 mmole) was added slowly, and the solution was stirred at room temperature for 15 min. Propargyl bromide (7.68 ml, 101.4 mmole) was added dropwise to the mixture at 0° C. The mixture was heated to 50° C. and stirred for another 16 hours. The obtained mixture was poured over ice/chloroform and the organic layer was collected and washed three times with brine. The organic layer was combined and dried over sodium sulfite, then filtered and concentrated by rotary evaporation. The crude product was washed with diethyl ether and isolated as dark yellow solid (18.5 g, 62.9 mmol, 80% yield). ¹H NMR (CDCl₃, 500 MHz, 298 K): δ_{H} 7.96 (d, J=9.0 Hz, 2H, Ar H), 7.25 (d, J=8.7 Hz, 2H, Ar H), 6.86 (dd, J=13.1, 8.8 Hz, 4H, Ar H), 4.70 (t, J=7.3 Hz, 1H, CH₂), 3.83 (s, 3H, OCH₃), 3.77 (s, 3H, OCH₃), 3.0 (ddd, J=16.8, 7.2 Hz, 1H, CH₂), 2.69 (ddd, J=18.2, 8.9, Hz, 1H, CH₂), 1.95 (t, J=2.6, Hz, 1H, CH). ¹³C NMR (CDCl₃, 125 MHz, 298 K): δ_{H} 196.57, 163.47, 158.94, 131.14, 130.64, 129.20,129.12, 114.43, 113.79, 82.79, 70.09, 55.31, 55.03, 53.73, 51.61, 23.43 ppm.

Synthesis of tetramethoxy deoxybenzoins triazole (TMDBT). 1,4-Bis(azidomethyl)benzene (2.00 g, 10.6 mmol) and alkynyl desoxyanisoin (7.80 g, 26.6 mmole) were combined as a solution in THF (20 mL) under N_{2(g)} atm. An aqueous solution (10 mL) of sodium ascorbate (1.57 g, 7.96 mmol, 0.30 eq) and CuSO₄·5H₂O (0.66 g, 2.65 mmol, 0.1 eq) was added immediately into the reaction mixture, which was degassed several times with N_{2(g)} and then heated to 30° C. for 12 h under an atmosphere of N_{2(g)}. The reaction mixture was extracted using dichloromethane and the organic liquid was dried over magnesium sulphate, then filtered and concentrated by rotary evaporation. The crude product was washed with ethyl acetate and diethyl ether, and the precipitate was filtered and finally obtained as a solid yellow powder (7.0 g, 9.01 mmol, 85% yield). ¹H NMR (CDCl₃, 500 MHz, 298 K): δ_{H} 7.92 (d, J=8.7 Hz, 4H, Ar H), 7.19 (s, 4H, Ar H), 7.05 (s, 6H Ar H), 6.84 (d, J=8.1 Hz, 4H, Ar H), 6.77 (m, 4H, Ar H), 5.42 (m, 4H CH₂), 5.03 (s, 2H CH₂), 3.82 (s, 3H, OCH₃), 3.74 (s, 3H, OCH₃), 3.55 (s, 2H, CH₂), 3.15 (s, 2H, CH₂). MALDI-TOF (ve⁺): m/z [M+H]⁺ calcd for C₄₆H₄₄N₆O₆=776.33, found: =776.57 [M+H]⁺. ¹³C NMR (CDCl₃, 125 MHz, 298 K): δ_{H} 197.80, 163.36, 158.65, 135.35, 131.23, 131.08, 129.20, 128.19, 114.33, 113.70, 55.44, 55.19, 30.39 ppm.

Synthesis of tetrahydroxy deoxybenzoins triazole (THDBT). TMDBT (5.00 g, 6.43 mmole) and hydroiodic acid (14 mL) were added to glacial acetic acid (10 mL) and the mixture was heated to 140° C. with stirring for 7 hours. The mixture was cooled to room temperature and poured into water to obtain a yellow precipitate, which was filtered to yield a yellow powder (4.0 g, 5.55 mmol, 86% yield). ¹H NMR (DMSO-d⁶, 500 MHz, 298 K): 10.30 (s, 2H, Ar—OH), 9.29 (s, 2H, Ar—OH), 7.86 (d, J=8.4 Hz, 4H), 7.66 (s, 2H), 7.11-7.05 (7H), 6.76 (d, J=8.4 Hz, 4H), 6.62 (d, J=8.1 Hz, 4H), 5.45 (s, 4H), 5.01 (t, J=7.5 Hz, 2H), 3.35 (dd, J=14.7, 8.3 Hz, 2H), 2.92 (dd, J=14.8, 6.5 Hz, 2H). ¹³C NMR (DMSO-d⁶, 125 MHz, 298 K): δ_{H} 197.51, 162.38, 156.69, 145.67, 136.46, 131.61, 130.08, 129.49, 128.33, 128.06, 123.11, 115.92, 115.61, 52.60, 50.67, 30.01 ppm. MALDI-TOF (ve⁺): m/z [M+H]⁺ calcd for C₄₂H₃₆N₆O₆=720.27, found: =720.39 [M+H]⁺.

Synthesis of deoxybenzoins triazole (DBT). Bis-alkynyl deoxybenzoin (BADB) (0.10 g, 0.33 mmole) and 1,4-bis(azidomethyl)benzene (0.185 g, 0.984 mmol) were combined as a solution in DMF (5 mL) under N_{2(g)} atm. In a 10 mL vial, a catalytic solution of CuI (10 mg, 5 mol %) and pyridine (1 mL) was prepared and added to the reaction mixture immediately. The reaction mixture was degassed several times with N_{2(g)} then heated at 50° C. for 4 h under N_{2(g)}. The reaction mixture was added to MeOH, and the resulting precipitate was filtered and washed with MeOH again, then dried under vacuum to obtain a beige powder (65 mg, 30% yield). ¹H NMR (CDCl₃, 500 MHz, 298 K): δ_{H} 7.99 (d, J=8.7 Hz, 2H), 7.57 (d, J=6.5 Hz, 2H), 7.36-7.35 (m, 4H), 7.43-7.34 (m, 4H), 7.32-7.30 (4H), 7.18 (d, J=8.3 Hz, 2H), 7.02 (d, J=8.7 Hz, 2H), 6.92 (d, J=8.6 Hz, 2H), 5.57 (d, J=5.2 Hz, 4H), 5.27 (s, 2H), 5.20 (s, 2H), 4.38 (s, 4H), 4.18 (s, 2H). MALDI-TOF (ve⁺): m/z [M+H]⁺ calcd for C₃₆H₃₂N₁₂O₃=680.27, found: =680.47 [M+H]⁺.

Preparation of P1. Bis-alkynyl deoxybenzoin (BADB) (0.485 g, 1.59 mmole) and 1,4-bis(azidomethyl)benzene (0.30 g, 1.6 mmol) were combined in DMF (10 mL) under N₂ atm. Separately, in a 10 mL vial, a solution of CuI (30 mg, 5%) and pyridine (1 mL) was prepared, then added to the reaction mixture. The mixture was degassed several times with N_{2(g)}, then heated at 55° C. for 12 h under N_{2(g)} atm. Then, the mixture was poured into MeOH, and the resulting precipitate was collected by filtration, then washed with MeOH and CH₂Cl₂ and dried under vacuum to obtain the polymer product as a beige powder (0.38 g, 78%). ¹H NMR (DMSO-d⁶, 500 MHz, 298 K): δ_{H} 8.29 (2H), 8.01 (2H), 7.32 (4H), 7.21-7.08 (4H), 6.94 (2H), 5.60 (4H), 5.23 (s, 2H), 5.09 (s, 2H), 4.24 (2H). ¹³C NMR (DMSO-d⁶, 125 MHz, 298 K): δ_{H} 196.77, 162.32, 157.16, 136,37, 132.50, 131,20, 131.07, 129.93, 128.94, 128.12, 125.40, 125.15, 115.08, 115.02, 61.78, 61.49, 52.94, 44.08 ppm.

### Preparation of Polymer-THDBT Blends

PET-THDBT blends. In a 20 mL vial was prepared a solution of PET (0.5 g) and 15 or 30 wt % THDBT (relative to PET) in hexafluoroisopropanol (HFIP) (5 mL). The solvent was removed in vacuo and the solid dried under vacuum oven at 60° C. for 12 h.

g-PET-THDBT blends. In a 20 mL vial was prepared a solution of g-PET (0.5 g) and 15 or 30 wt % THDBT (relative to g-PET) in HFIP (5 mL). The solvent was removed in vacuo and the solid dried under vacuum oven at 60° C. for 12 h.

PBT-THDBT blends. In a 20 mL vial was prepared a solution of PBT (0.5 g) and 15 or 30 wt % THDBT (relative to PBT) in HFIP (5 mL). The solvent was removed in vacuo and the solid dried under vacuum oven at 60° C. for 12 h.

P4VP-THDBT blends. In a 20 mL vial was prepared a solution of P4VP (0.5 g) and 15 or 30 wt % THDBT (relative to P4VP) in MeOH (5 mL). The solvent was removed in vacuo and the solid dried under vacuum oven at 60° C. for 12 h.

PS-THDBT blends. In a 20 mL vial was prepared a solution of P4VP (0.5 g) and 15 or 30 wt % THDBT (relative to PS) in THF (5 mL). The solvent was removed in vacuo and the solid dried under vacuum oven at 60° C. for 12 h.

General procedure for fabrication of polymer/THDBT films. Samples for flame-retardant tests were prepared by solvent casting. In a typical example, a solution of PET (1.5 g) in hexafluoroisopropanol (HFIP) (10 ml) was prepared, to which was added 30 or 50 wt % THDBT (relative to PET) to give a homogeneous solution. The solution was poured on rectangular silicone mold and dried slowly at room temperature for 24 h, then at 60° C. in a convection oven for 24 h to afford a film of ˜3 mm thickness (measured with digital calipers). A similar procedure was employed for the preparation of g-PET/THDBT and PBT-THDBT films, using MeOH (10 mL) for P4VP-THDBT and THF (10 mL) for PS-THDBT. The solutions were poured on rectangular silicone mold and dried slowly at room temperature for 24 h, then dried in a convection oven at 30° C. for 48 h.

This disclosure further encompasses the following aspects.

Aspect 1: A deoxybenzoin compound having the structure (I), (II), (III), (IV), or (V)

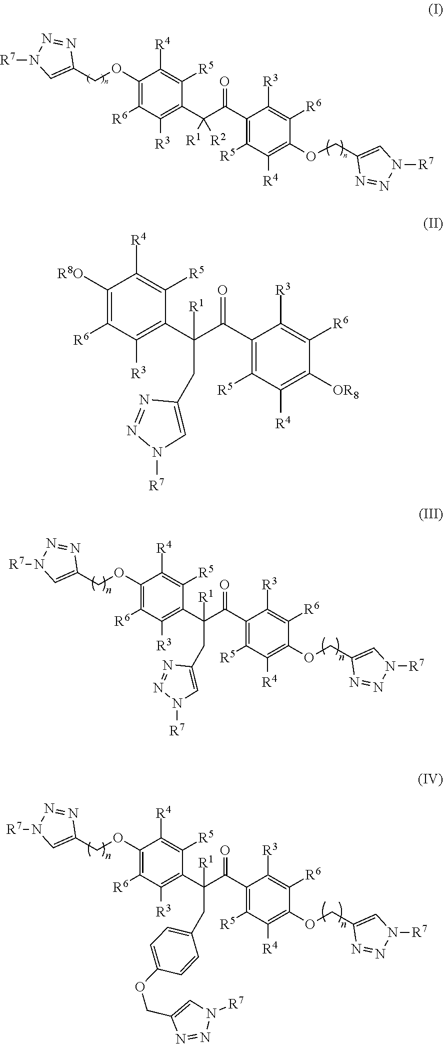

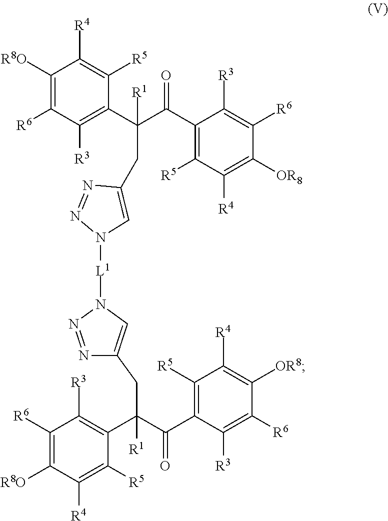

wherein in the foregoing formulas: R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; R⁷ is independently at each occurrence a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R⁸ is independently at each occurrence hydrogen or a C₁₋₆ alkyl group; L¹ is a linking group; and n is independently at each occurrence an integer from 1 to 12.

Aspect 2: The deoxybenzoin compound of aspect 1, wherein each occurrence of n is 1.

Aspect 3: The deoxybenzoin compound of aspect 1 or 2, wherein each occurrence of R¹ and R² is hydrogen.

Aspect 4: The deoxybenzoin compound of any of aspects 1 to 3, wherein each occurrence of R³, R⁴, R⁵, and R⁶ is hydrogen.

Aspect 5: The deoxybenzoin compound of aspect 1, wherein the deoxybenzoin compound has the structure (I)

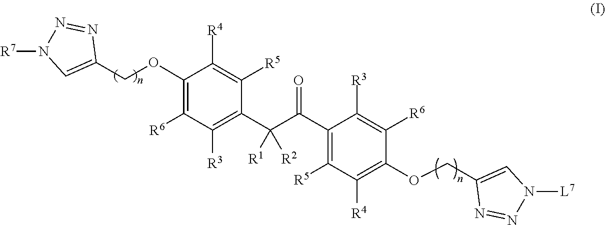

wherein each occurrence of R¹ and R² is hydrogen; each occurrence of R³, R⁴, R⁵, and R⁶ is hydrogen; each occurrence of n is 1; and R⁷ is C₇₋₁₃ alkylaryl group, preferably a benzyl group.

Aspect 6: The deoxybenzoin compound of aspect 1, wherein the deoxybenzoin compound has the structure (II)

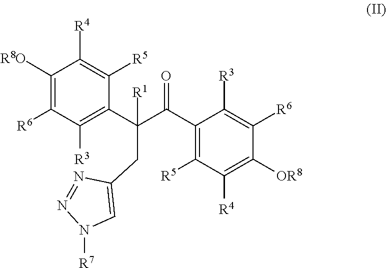

wherein R¹ is hydrogen; each occurrence of R³, R⁴, R⁵, and R⁶ is hydrogen; R⁷ is C₇₋₁₃ alkylaryl group, preferably a benzyl group; and R⁸ is hydrogen or methyl.

Aspect 7: The deoxybenzoin compound of aspect 1, wherein the deoxybenzoin compound has the structure (III)

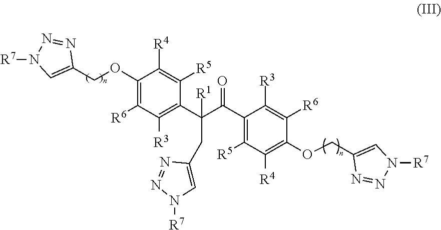

wherein R¹ is hydrogen; each occurrence of R³, R⁴, R⁵, and R⁶ is hydrogen; each occurrence of R⁷ is C₇₋₁₃ alkylaryl group, preferably a benzyl group; and n is 1.

Aspect 8: The deoxybenzoin compound of aspect 1, wherein the deoxybenzoin compound has the structure (IV)

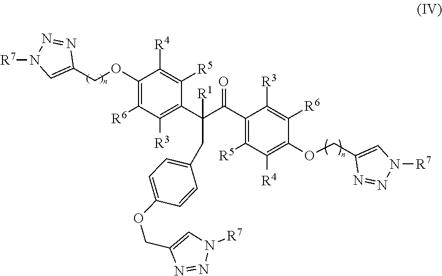

wherein R¹ is hydrogen; each occurrence of R³, R⁴, R⁵, and R⁶ is hydrogen; each occurrence of R⁷ is C₇₋₁₃ alkylaryl group, preferably a benzyl group; and n is 1.

Aspect 9: The deoxybenzoin compound of aspect 1, wherein the deoxybenzoin compound has the structure (V)

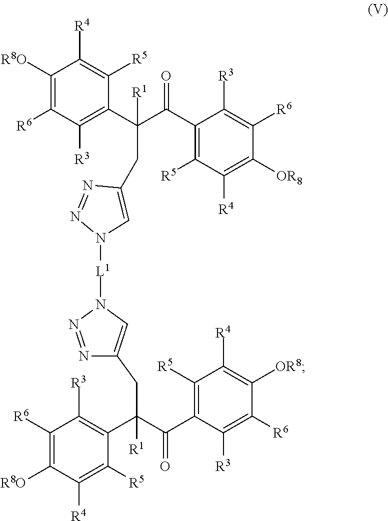

wherein each occurrence of R¹ is hydrogen; each occurrence of R³, R⁴, R⁵, and R⁶ is hydrogen; R⁸ is hydrogen or methyl; and L¹ is a C₁₋₆ alkylene group, a C₆₋₂₀ arylene group, a C₇₋₁₃ arylalkylene group, or a C₇₋₁₃ alkylarylene group.

Aspect 10: The deoxybenzoin compound of any of aspects 1 to 9, wherein the deoxybenzoin compound exhibits one or more of the following properties: a heat release capacity of less than or equal to 325 joules per gram-Kelvin, determined using a pyrolysis combustion flow calorimeter; a total heat release of less than 20 kilojoules per gram, determined using a pyrolysis combustion flow calorimeter; or a char yield of at least 20 percent, after 60 minutes at 800° C., as determined by thermogravimetric analysis.

Aspect 11: A composition comprising the deoxybenzoin compound of any of aspects 1 to 10, preferably wherein the composition comprises a thermoplastic polymer; more preferably wherein the composition comprises: 0.01 to 50 weight percent of the deoxybenzoin compound; and 50 to 99.99 weight percent of the thermoplastic polymer.

Aspect 12: A polymer comprising repeating units of the formula

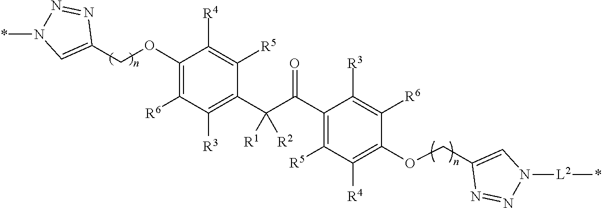

wherein: R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group; R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group; L² is independently at each occurrence a linking group; and n is independently at each occurrence an integer from 1 to 12.

Aspect 13: The polymer of aspect 12, wherein each occurrence of n is 1.

Aspect 14: The polymer of aspect 12 or 13, wherein each occurrence of R¹ and R² is hydrogen.

Aspect 15: The polymer of any of aspects 12 to 14, wherein each occurrence of R³, R⁴, R⁵, and R⁶ is hydrogen.

Aspect 16: The polymer of any of aspect 12 to 15, wherein L² is a C₁₋₆ alkylene group, a C₆₋₂₀ arylene group, a C₇₋₁₃ arylalkylene group, or a C₇₋₁₃ alkylarylene group.

Aspect 17: The polymer of any of aspects 12 to 16, wherein the polymer exhibits one or more of the following properties: a heat release capacity of less than or equal to 150 joules per gram-Kelvin, determined using a pyrolysis combustion flow calorimeter; a total heat release of less than 20 kilojoules per gram, determined using a pyrolysis combustion flow calorimeter; a char yield of at least 20 percent after 60 minutes at 800° C., as determined by thermogravimetric analysis; or a fire growth capacity of less than 50 joules per gram-Kelvin.

Aspect 18: The polymer of any of aspects 12 to 17, wherein the polymer has a molecular weight of 1,000 to 500,000 grams per mole.

Aspect 19: The polymer of any of aspects 12 to 18, wherein the polymer is at least partially crosslinked.

Aspect 20: An article comprising the polymer of any of aspects 12 to 19.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

As used herein, the term “hydrocarbyl”, whether used by itself, or as a prefix, suffix, or fragment of another term, refers to a residue that contains only carbon and hydrogen. The residue can be aliphatic or aromatic, straight-chain, cyclic, bicyclic, branched, saturated, or unsaturated. It can also contain combinations of aliphatic, aromatic, straight chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. The term “alkyl” means a branched or straight chain, saturated aliphatic hydrocarbon group, e.g., methyl, ethyl, n-propyl, i-propyl, n-butyl, s-butyl, t-butyl, n-pentyl, s-pentyl, and n-and s-hexyl. “Alkenyl” means a straight or branched chain, monovalent hydrocarbon group having at least one carbon-carbon double bond (e.g., ethenyl (—HC═CH₂)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)). “Cycloalkylene” means a divalent cyclic alkylene group, —CₙH₂ₙ₋ₓ, wherein x is the number of hydrogens replaced by cyclization(s). “Cycloalkenyl” means a monovalent group having one or more rings and one or more carbon-carbon double bonds in the ring, wherein all ring members are carbon (e.g., cyclopentyl and cyclohexyl). “Aryl” means an aromatic hydrocarbon group containing the specified number of carbon atoms, such as phenyl, tropone, indanyl, or naphthyl. “Arylene” means a divalent aryl group. “Alkylarylene” means an arylene group substituted with an alkyl group. “Arylalkylene” means an alkylene group substituted with an aryl group (e.g., benzyl). The prefix “halo” means a group or compound including one more of a fluoro, chloro, bromo, or iodo substituent. A combination of different halo atoms (e.g., bromo and fluoro), or only chloro atoms can be present. The prefix “hetero” means that the compound or group includes at least one ring member that is a heteroatom (e.g., 1, 2, or 3 heteroatom(s)), wherein the heteroatom(s) is each independently N, O, S, Si, or P. “Substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl), a thiol (—SH), a thiocyano (—SCN), a tosyl (CH₃C₆H₄SO₂—), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a nitrile.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A deoxybenzoin compound having the structure (I), (II), (III), (IV), or (V) 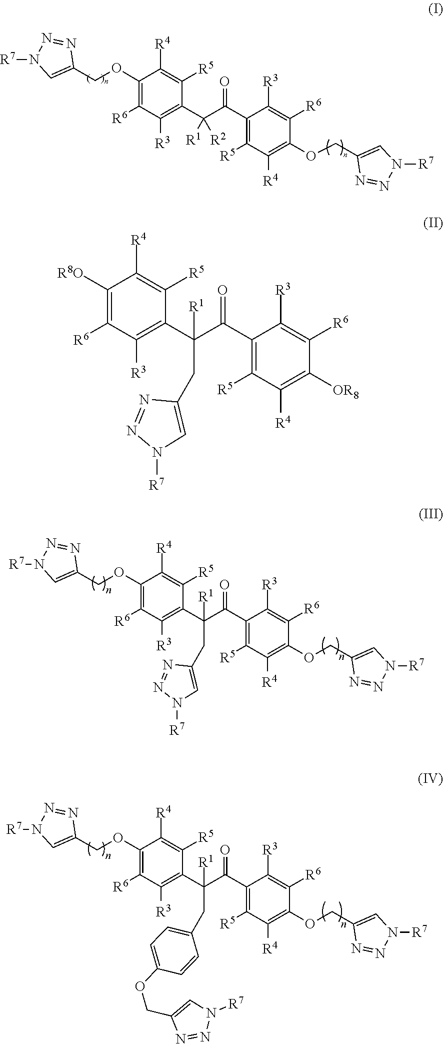 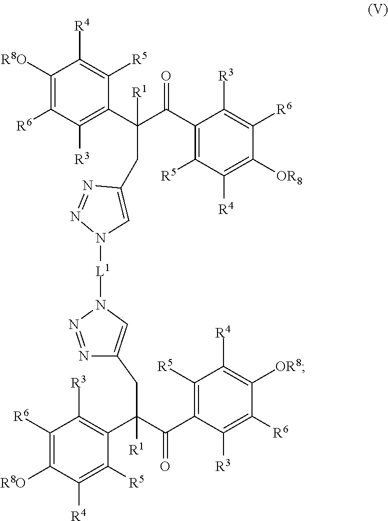
wherein in the foregoing formulas:
R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group;
R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group;
R⁷ is independently at each occurrence a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group;
R⁸ is independently at each occurrence hydrogen or a C₁₋₆ alkyl group;
L¹ is a linking group; and
n is independently at each occurrence an integer from 1 to 12.

2. The deoxybenzoin compound of claim 1, wherein each occurrence of n is 1.

3. The deoxybenzoin compound of claim 1, wherein each occurrence of R¹ and R² is hydrogen.

4. The deoxybenzoin compound of claim 1, wherein each occurrence of R³, R⁴, R⁵, and R⁶ is hydrogen.

5. The deoxybenzoin compound of claim 1, wherein the deoxybenzoin compound has the structure (I) 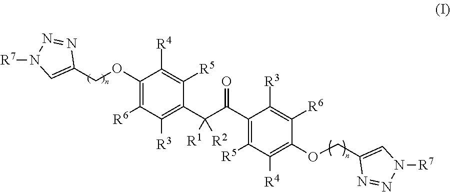
wherein
each occurrence of R¹ and R² is hydrogen;
each occurrence of R³, R⁴, R⁵, and R⁶ is hydrogen;
each occurrence of n is 1; and
R⁷ is C₇₋₁₃ alkylaryl group.

6. The deoxybenzoin compound of claim 1, wherein the deoxybenzoin compound has the structure (II) 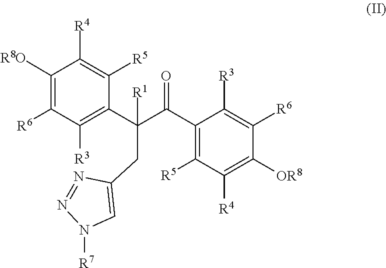
wherein
R¹ is hydrogen;
each occurrence of R³, R⁴, R⁵, and R⁶ is hydrogen;
R⁷ is C₇₋₁₃ alkylaryl group; and
R⁸ is hydrogen or methyl.

7. The deoxybenzoin compound of claim 1, wherein the deoxybenzoin compound has the structure (III) 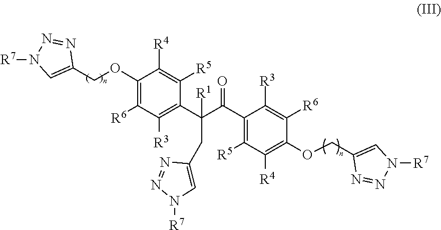
wherein
R¹ is hydrogen;
each occurrence of R³, R⁴, R⁵, and R⁶ is hydrogen;
each occurrence of R⁷ is C₇₋₁₃ alkylaryl group; and
n is 1.

8. The deoxybenzoin compound of claim 1, wherein the deoxybenzoin compound has the structure (IV) 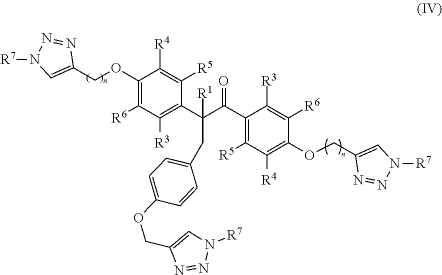
wherein
R¹ is hydrogen;
each occurrence of R³, R⁴, R⁵, and R⁶ is hydrogen;
each occurrence of R⁷ is C₇₋₁₃ alkylaryl group; and
n is 1.

9. The deoxybenzoin compound of claim 1, wherein the deoxybenzoin compound has the structure (V) 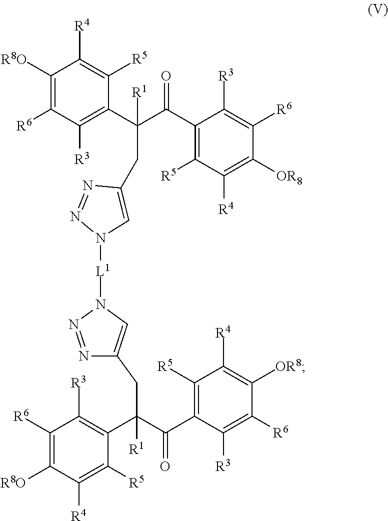
wherein
each occurrence of R¹ is hydrogen;
each occurrence of R³, R⁴, R⁵, and R⁶ is hydrogen;
R⁸ is hydrogen or methyl; and
L¹ is a C₁₋₆ alkylene group, a C₆₋₂₀ arylene group, a C₇₋₁₃ arylalkylene group, or a C₇₋₁₃ alkylarylene group.

10. The deoxybenzoin compound of claim 1, wherein the deoxybenzoin compound exhibits one or more of the following properties:
a heat release capacity of less than or equal to 325 joules per gram-Kelvin, determined using a pyrolysis combustion flow calorimeter;
a total heat release of less than 20 kilojoules per gram, determined using a pyrolysis combustion flow calorimeter; or
a char yield of at least 20 percent, after 60 minutes at 800° C., as determined by thermogravimetric analysis.

11. A composition comprising the deoxybenzoin compound of claim 1, wherein the composition further comprises a thermoplastic polymer.

12. A polymer comprising repeating units of the formula 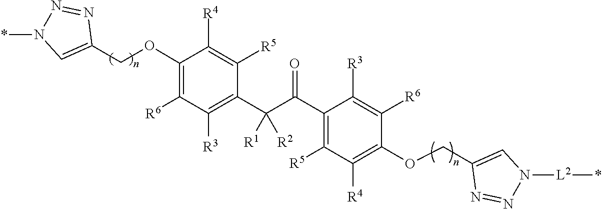
wherein:
R¹ and R² are independently at each occurrence hydrogen, a halogen, a nitrile group, a C₁₋₆ alkyl group, a C₂₋₁₃ alkenyl group, a C₂₋₁₃ alkynyl group, a C₆₋₂₀ aryl group, a C₇₋₁₃ arylalkyl group, or a C₇₋₁₃ alkylaryl group;
R³, R⁴, R⁵, and R⁶ are independently at each occurrence hydrogen, a halogen, a hydroxyl group, a nitrile group, a C₁₋₆ alkyl group, a C₁₋₆ alkoxy group, or a C₆₋₂₀ aryl group;
L² is independently at each occurrence a linking group; and
n is independently at each occurrence an integer from 1 to 12.

13. The polymer of claim 12, wherein each occurrence of n is 1.

14. The polymer of claim 12, wherein each occurrence of R¹ and R² is hydrogen.

15. The polymer of claim 12, wherein each occurrence of R³, R⁴, R⁵, and R⁶ is hydrogen.

16. The polymer of claim 12, wherein L² is a C₁₋₆ alkylene group, a C₆₋₂₀ arylene group, a C₇₋₁₃ arylalkylene group, or a C₇₋₁₃ alkylarylene group.

17. The polymer of claim 12, wherein the polymer exhibits one or more of the following properties:
a heat release capacity of less than or equal to 150 joules per gram-Kelvin, determined using a pyrolysis combustion flow calorimeter;
a total heat release of less than 20 kilojoules per gram, determined using a pyrolysis combustion flow calorimeter;
a char yield of at least 20 percent after 60 minutes at 800° C., as determined by thermogravimetric analysis; or
a fire growth capacity of less than 50 joules per gram-Kelvin.

18. The polymer of claim 12, wherein the polymer has a molecular weight of 1,000 to 500,000 grams per mole.

19. The polymer of claim 12, wherein the polymer is at least partially crosslinked.

20. An article comprising the polymer of claim 12.
